(19) United States
(12) Patent Application Publication
(10) Pub. No.: US 2020/0289648 A1
(43) Pub. Date: Sep. 17, 2020

(54) COMPLEX WITH CORE-SHELL STRUCTURE AND APPLICATIONS THEREOF

(71) Applicant: Taipei Medical University, Taipei City (TW)

(72) Inventors: Er-Yuan CHUANG, Taipei City (TW); Chih-Hwa CHEN, Taipei City (TW); Chih-Wei CHIANG, Taipei City (TW); Pei-Ru JHENG, Taipei City (TW); Nyambat BATZAYA, Taipei City (TW); Mantosh Kumar SATAPATHY, Taipei City (TW); Shao-Chan HUANG, Taipei City (TW); Er-Chen CHO, Taipei City (TW); Ting-Han CHEN, Taipei City (TW)

(21) Appl. No.: 16/802,607

(22) Filed: Feb. 27, 2020

(30) Foreign Application Priority Data
Mar. 14, 2019 (TW) 108108701

(51) Int. Cl. A61K 41/00; A61K 9/48; A61P 7/02; A61P 35/00; A61H 39/06; A61N 5/06; A61F 7/02; D06M 16/00

(52) U.S. Cl. CPC A61K 41/0052; A61K 9/4866; A61K 9/4816; A61P 7/02; A61P 35/00; D06M 2101/20; A61H 39/06; A61N 5/0625; A61F 7/02; D06M 16/00; A61F 2007/0287; A61K 9/4808

(57) ABSTRACT

The present disclosure relates to a complex having a core-shell structure, a composition and a textile comprising the same, and a method for treating thrombosis, cancer, or wounds using the same. The complex having the core-shell structure comprises a core; and a shell layer covering a surface of the core; wherein the core is made of polypyrrole.

### CROSS REFERENCE TO RELATED APPLICATION

This application claims the benefits of the Taiwan Patent Application Ser. No. 108108701, filed on Mar. 14, 2019, the subject matter of which is incorporated herein by reference.

### BACKGROUND OF THE INVENTION

### 1. Field of the Invention

The present disclosure relates to a complex and, in particular, to a complex having a core-shell structure.

### 2. Description of Related Art

Polypyrrole (Ppy) is a bioorganic conducting polymer which has long been recognized as a versatile material used in display, photoelectric and semiconductor industries owing to its excellent stability, conductive properties, and great absorbance in the range of near-infrared (NIR). However, polypyrrole has limited applications in the biomedical field due to its hydrophobicity.

Currently, in the field of medical technology, chemotherapy has been mainly used for cancer in these middle and later stage and is able to be administered before or after medical resection, in place of surgery when the tumorous region is unresectable. However, chemotherapy would cause a drug sensitized response, low bioavailability, and other harmful side effects. Since systemic chemotherapy intravenously administrated is not exclusively distributed to the tumorous site, it is actually hard to attain beneficial dosage levels of active medicine inside or around the tumorous region. Moreover, a substantial amount of active medicine regularly accumulates within normal tissues, causing toxic reactions and undesired side effects. Therefore, patients need to endure the discomfort caused by chemotherapy while being treated.

Further, mesh dressings are commonly used for wound healing. Although mesh dressings are convenient and cheap, they are adherent to the wound, which causes pain while mesh dressings are changed. In addition, mesh dressings are not completely attached to the damaged wound surface, and cannot promote epidermal cell migration and wound healing

In addition, common treatments for venous or arterial thrombosis include administration of thrombolytic agents, for instance streptokinase. However, the administration of thrombolytic agents can induce life-threatening bleeding syndromes and bring great risk to patients while treating thrombosis.

Furthermore, the heat-preservation ability of a textile is one of the most vital considerations influencing the thermal comfort provided by the textile upon wearing. The precise determination of the heat-preservation ability of the textile is the key to selecting the clothing and fabric for various end consumers, designing of functionality clothing, and environmental thermal engineering. However, existing textiles do not provide adequate heat-preservation ability.

In view of the above, there is an urgent need to develop a versatile material that can be applied in the field of biomedicine or textile to effectively treat cancer, thrombosis or wounds or provide excellent heat-preservation ability, so that the discomfort accompanied with cancer treatment or bleeding caused during thrombosis treatment can be avoided, wounds can heal faster, or the thermal functionality of a textile can be improved.

### SUMMARY OF THE INVENTION

To solve the above issues, the present disclosure provide a complex having a core-shell structure to effectively treat cancer, thrombosis or wounds or provide excellent heat-preservation ability, so that the discomfort accompanied with cancer treatment or bleeding caused during thrombosis treatment can be avoided, wounds can heal faster, or the thermal functionality of a textile can be improved.

In one aspect of the present disclosure, there is provided a complex having a core-shell structure, wherein the complex comprises: a core; and a shell layer covering a surface of the core; wherein the core is made of polypyrrole (Ppy).

In an embodiment of the complex according to the present disclosure, the shell layer is preferably made of an amphiphilic polymer; more preferably, the shell layer is made of a material selected from the group consisting of polyethylenimine (PEI), heparin, fucoidan, hyaluronic acid, glyco chitosan, and a combination thereof; and even more preferably, the shell layer is made of PEI.

In an embodiment of the complex according to the present disclosure, the complex preferably has a size ranging from 10 nm to 1500 nm; more preferably 15 nm to 1000 nm; and most preferably 20 nm to 500 nm.

In an embodiment of the complex according to the present disclosure, a weight ratio of the shell layer to the core is not limited, and is preferably from 1500: 500 to 100: 4 and more preferably from 300: 50 to 100: 5.

In another aspect of the present disclosure, there is provided a method for preparing a complex having a core-shell structure. The method comprises the following steps: (A) providing a solution of polyethylenimine dissolved in water; (B) adding a pyrrole monomer into the solution; and (C) adding a catalyst into the solution to form a mixture.

In an embodiment of the method for preparing a complex having a core-shell structure according to the present disclosure, step (B) may further comprise mixing under any pH value. Preferably, the pH value is less than 1.2. More preferably, the pH value ranges from 0.5 to 1.2.

In an embodiment of the method for preparing a complex having a core-shell structure according to the present disclosure, the catalyst may be ferric chloride hexahydrate.

In an embodiment of the method for preparing a complex having a core-shell structure according to the present disclosure, a weight ratio of the polyethylenimine to the pyrrole monomer is not limited, and is preferably from 1500:500 to 100:4 and more preferably from 300:50 to 100:5.

In an embodiment of the method for preparing a complex having a core-shell structure according to the present disclosure, the method further comprises a step (D): eliminating residual polyethylenimine and the catalyst from the mixture. Herein, a dialysis process may be performed on the mixture to eliminate the residual polyethylenimine and the catalyst. In particular, the dialysis process can be performed with a dialysis bag.

In another aspect of the present invention, there is provided a method for treating thrombosis. The method comprises: administrating to a subject in need thereof an effective amount of a complex having a core-shell structure, and the complex comprises: a core; and a shell layer covering a surface of the core; wherein the core is made of polypyrrole.

In an embodiment of the method for treating thrombosis according to the present disclosure, the shell layer is preferably made of an amphiphilic polymer; more preferably, the shell layer is made of a material selected from the group consisting of polyethylenimine (PEI), heparin, fucoidan, hyaluronic acid, glyco chitosan, and a combination thereof; and even more preferably, the shell layer is made of PEI.

In an embodiment of the method for treating thrombosis according to the present disclosure, the complex preferably has a size ranging from 10 nm to 1500 nm; more preferably 15 nm to 1000 nm; and most preferably 20 nm to 500 nm.

In an embodiment of the method for treating thrombosis according to the present disclosure, a weight ratio of the shell layer to the core is preferably from 1500:500 to 100:4, and more preferably from 300:50 to 100:5.

In another aspect of the present invention, there is provided a method for treating cancer. The method comprises: administrating to a subject in need thereof an effective amount of a complex having a core-shell structure, and the complex comprises: a core; and a shell layer covering a surface of the core; wherein the core is made of polypyrrole.

In an embodiment of the method for treating cancer according to the present disclosure, the shell layer is preferably made of an amphiphilic polymer; more preferably, the shell layer is made of a material selected from the group consisting of polyethylenimine (PEI), heparin, fucoidan, hyaluronic acid, glyco chitosan, and a combination thereof; and even more preferably, the shell layer is made of PEI.

In an embodiment of the method for treating cancer according to the present disclosure, the complex preferably has a size ranging from 10 nm to 1500 nm; more preferably 15 nm to 1000 nm; and most preferably 20 nm to 500 nm.

In an embodiment of the method for treating cancer according to the present disclosure, a weight ratio of the shell layer to the core is preferably from 1500:500 to 100:4, and more preferably from 300:50 to 100:5.

In an embodiment of the method for treating cancer according to the present disclosure, the cancer may be any type of cancer. Preferably, the cancer is lung cancer.

In another aspect of the present invention, there is provided a composition, comprising: a complex having a core-shell structure, and a polymer; wherein the complex comprises: a core made of polypyrrole; and a shell layer covering a surface of the core.

In an embodiment of the composition according to the present disclosure, the shell layer is preferably made of an amphiphilic polymer; more preferably, the shell layer is made of a material selected from the group consisting of polyethylenimine (PEI), heparin, fucoidan, hyaluronic acid, glyco chitosan, and a combination thereof, and even more preferably, the shell layer is made of PEI.

In an embodiment of the composition according to the present disclosure, the complex preferably has a size ranging from 10 nm to 1500 nm; more preferably 15 nm to 1000 nm; and most preferably 20 nm to 500 nm.

In an embodiment of the composition according to the present disclosure, a weight ratio of the shell layer to the core is not limited, and is preferably from 1500:500 to 100:4 and more preferably from 300:50 to 100:5.

In an embodiment of the composition according to the present disclosure, the polymer may be a hydrogel or a binder. Preferably, the polymer may be a thermally sensitive hydrogel or a vinyl acrylate binder.

In another aspect of the present invention, there is provided a method for treating a wound. The method comprises: administrating to a subject in need thereof an effective amount of a composition comprising a complex having a core-shell structure, wherein the complex comprises: a core made of polypyrrole; and a shell layer covering a surface of the core.

In an embodiment of the method for treating a wound according to the present disclosure, the composition further comprises a hydrogel. Preferably, the composition further comprises a thermally sensitive hydrogel.

In an embodiment of the method for treating a wound according to the present disclosure, the shell layer is preferably made of an amphiphilic polymer; more preferably, the shell layer is made of a material selected from the group consisting of polyethylenimine (PEI), heparin, fucoidan, hyaluronic acid, glyco chitosan, and a combination thereof; and even more preferably, the shell layer is made of PEI.

In an embodiment of the method for treating a wound according to the present disclosure, preferably, the wound is a skin wound.

In another aspect of the present invention, there is provided a textile, which comprises: a fiber; and a complex having a core-shell structure and attached to the fiber; wherein the complex comprises: a core made of polypyrrole; and a shell layer covering a surface of the core.

In an embodiment of the textile according to the present disclosure, the fiber can be any fiber. For example, the fiber can be cotton fibers, linen fibers, wool fibers, silk fibers, man-made fibers (such as polypropylene (PP), polyethylene (PE), polystyrene (PS), polyurethane (PU), polyacrylic acid (PAA), polyester (PET) or nylon fibers), or a combination thereof In one embodiment of the present disclosure, the fiber is a polyethylene (PE) fiber.

In an embodiment of the textile according to the present disclosure, the complex may be attached to the fiber by any method. Preferably, the complex is attached to the fiber by dip-coating. Alternatively, the complex may be attached to the fiber using a binder by printing, and the binder is preferably a vinyl acrylate binder.

In an embodiment of the textile according to the present disclosure, the shell layer is preferably made of an amphiphilic polymer; more preferably, the shell layer is made of a material selected from the group consisting of polyethylenimine (PEI), heparin, fucoidan, hyaluronic acid, glyco chitosan, and a combination thereof; and even more preferably, the shell layer is made of PEI.

In an embodiment of the textile according to the present disclosure, the complex preferably has a size ranging from 10 nm to 1500 nm; more preferably 15 nm to 1000 nm; and most preferably 20 nm to 500 nm.

In an embodiment of the textile according to the present disclosure, a weight ratio of the shell layer to the core is not limited, and is preferably from 1500:500 to 100:4 and more preferably from 300:50 to 100:5.

The terms “treatment”, “under treatment” and “therapy” used in the present disclosure include alleviating, mitigating, or improving at least one disease symptom or physiological condition, preventing new symptoms, suppressing diseases or a physiological condition, preventing or slowing the development of a disease, causing the recovery of a disease or a physiological condition, slowing a physiological condition caused by a disease, stopping a disease symptom or a physiological condition by means of treatment or prevention.

The term “an effective amount” refers to the amount of the complex or composition which is required to confer the desired effect on the subject. Effective amounts vary, as recognized by those skilled in the art, depending on route of administration, excipient usage, and the possibility of co-usage with other therapeutic treatments such as use of other active agents.

Other objects, advantages, and novel features of the invention will become more apparent from the following detailed description when taken in conjunction with the accompanying drawings.

### BRIEF DESCRIPTION OF THE DRAWINGS

FIG. 1 is a schematic diagram illustrating a core-shell structure of Ppy-PEI NC obtained from an embodiment of the present disclosure;

FIG. 2 shows SEM images of pure gelatin hydrogel and Ppy-PEI NC hydrogel obtained from an embodiment of the present disclosure;

FIG. 3A shows thermal images of a near-infrared (NIR) irradiated Ppy-PEI NC hydrogel according to an embodiment of the present disclosure;

FIG. 3B is a graph showing the quantitative temperature variation of FIG. 3A;

FIG. 4 is a schematic flow diagram illustrating application of the Ppy-PEI NC hydrogel onto a skin wound according to an embodiment of the present disclosure;

FIG. 5 shows the MTT assay results of the Ppy-PEI NC hydrogel according to an embodiment of the present disclosure;

FIG. 6A shows the macroscopic images of rat skin wounds applied with the Ppy-PEI NC hydrogel according to an embodiment of the present disclosure;

FIG. 6B shows the quantitative percentage of wound contraction (%);

FIG. 6C shows images of histological sections of primary organs harvested from the tested rats;

FIG. 6D is a graph showing percentage change in body weight (%) of the tested rats;

FIG. 7A shows photographs and images of an aqueous Ppy-PEI NC solution and an aqueous Ppy solution captured by a general camera or transmission electron microscopy (TEM);

FIG. 7B shows an SEM image of the aqueous Ppy-PEI NC solution according to an embodiment of the present disclosure;

FIG. 8A shows images of Ppy-PEI NC incubated respectively with gelatin (A) and gelatin (B) hydrogels captured by confocal laser scanning microscopy (CLSM);

FIG. 8B is a graph showing the analyzed result of the Ppy-PEI NC by Fourier transform infrared (FTIR) spectroscopy;

FIG. 9A shows fluorescent confocal laser scanning microscopy images illustrating cell endocytosis and detected ROS according to an embodiment of the present disclosure;

FIG. 9B is a graph showing statistical analysis results of ROS fluorescence intensity in FIG. 9A;

FIG. 9C shows fluorescent confocal laser scanning microscopy images illustrating cell endocytosis and detected H₂O₂ according to an embodiment of the present disclosure;

FIG. 9D is a graph showing statistical analysis results of H₂O₂ fluorescence intensity in FIG. 9C;

FIG. 10A is a graph showing statistical analysis results of MTT assays according to an embodiment of the present disclosure;

FIG. 10B shows SEM images of NCI-H460 cancer cells according to an embodiment of the present disclosure;

FIG. 11 shows images of cells captured by fluorescence microscopy;

FIG. 12 shows CLSM images of colt morphology, illustrating the in vitro anti-clot effect of Ppy-PEI NC with NIR irradiation according to an embodiment of the present disclosure; FIG. 13 shows SEM images of macrophages co-localized with the positively charged Cy5-Ppy-PEI NC according to an embodiment of the present disclosure;

FIG. 14 shows IVIS images of the thrombus sites at femur veins of Wistar rats according to an embodiment of the present disclosure;

FIG. 15A shows IVIS images of rats' feet according to an embodiment of the present disclosure;

FIG. 15B shows images of rats' feet captured by a thermal camera according to an embodiment of the present disclosure;

FIG. 15C shows images of tissue sections captured by optical microscopy according to an embodiment of the present disclosure;

FIG. 15D shows images of organ sections captured by optical microscopy according to an embodiment of the present disclosure;

FIG. 16A shows SEM images of PEFM and Ppy-PEI NC-PEFM according to an embodiment of the present disclosure;

FIG. 16B shows microscopic images and MD simulation of Ppy-PEI NC-PEFM according to an embodiment of the present disclosure;

FIG. 16C shows photographs of Ppy-PEI NC-PEFM before and after washing according to an embodiment of the present disclosure;

FIG. 16D shows wavelength of NIR lamp light determined by a spectrometer;

FIG. 16E shows thermal images of PEFM and Ppy-PEI NC-PEFM before and after washing captured by a thermal camera according to an embodiment of the present disclosure;

FIG. 16F is a graph showing the thermal analysis results of PEFM and Ppy-PEI NC-PEFM before and after washing according to an embodiment of the present disclosure;

FIG. 16G is a graph showing the temperature change of repeated NIR irradiation on Ppy-PEI NC-PEFM detected by a thermocouple;

FIG. 17A shows In-vitro biocompatibility test results of MTT assay according to an embodiment of the present disclosure;

FIG. 17B shows images of live/dead cells captured by fluorescence microscopy according to an embodiment of the present disclosure;

FIG. 18A shows fluorescence images of PEFM and Cy5-Ppy-PEI NC-PEFM as well as their statistical analysis by ImageJ software;

FIG. 18B shows fluorescence images of E. coli bacteria treated PEFM and Cy5-Ppy-PEI NC-PEFM captured by fluorescence microscopy as well as their statistical analysis by ImageJ software;

FIG. 18C shows fluorescence images of E. coli bacteria treated Cy5-Ppy-PEI NC-PEFM before and after NIR irradiation captured by fluorescence microscopy as well as their statistical analysis by ImageJ software;

FIG. 19 shows thermal images of the tested rats' backs;

FIG. 20A shows images of skin sections captured by optical microscopy according to an embodiment of the present disclosure; and

FIG. 20B shows images of organ sections captured by optical microscopy according to an embodiment of the present disclosure.

### DETAILED DESCRIPTION OF THE PREFERRED EMBODIMENT

The implementations of the present disclosure will be described with specific embodiments in the following description. A person skilled in the art will understand the advantages and the effects provided by the present disclosure. Different specific embodiments may be applicable according to the present disclosure.

The Ppy-PEI NC (polypyrrole-polyethylenimine nanocomplex) of the present disclosure is prepared by dissolving PEI (600 Da, 20-2000 mg) in deionized (DI) water to form a solution, into which a pyrrole monomer (1-200 μL) is then added. The resulting solution is then stirred for 0.2-3 h at specific pH. Subsequently, ferric chloride hexahydrate (0.0005-0.1g/mL, 0.1-10 mL) is added into the solution. After 0.1-2 h of polymerization, a dialysis bag is used to eliminate free PEI and ferric ions. Afterwards, DI water washing (3-30 times) and then oven drying (about 1-7 days) are performed to obtain Ppy-PEI NC (20-1000 nm).

### PREPARATION EXAMPLE 1

### Preparation of Ppy-PEI NC hydrogel

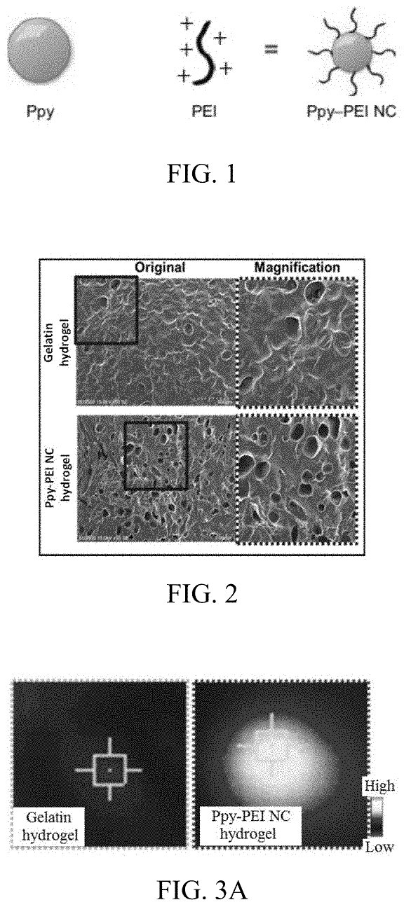

FIG. 1 is a schematic view showing a core-shell structure of Ppy-PEI NC obtained from an embodiment of the present disclosure. PEI (600 Da, 200 mg, purchased from Sigma-Aldrich) was dissolved in 20 mL of DI water to form a solution, into which a pyrrole monomer (12.5 μL, purchased from Sigma-Aldrich) was then added. The resulting solution was stirred for 0.2-3 h before addition of ferric chloride hexahydrate (12.5 mg/mL, 1 mL, purchased from Sigma-Aldrich). After 0.1-2 h of polymerization, a dialysis bag was used to eliminate free PEI and ferric ions, and then DI water washing and oven drying were performed to obtain Ppy-PEI NC (20-1000 nm).

To prepare the gelatin hydrogel containing the Ppy-PEI NC, the gelatin (type B, from bovine skin, purchased from Sigma-Aldrich) was dissolved in warm phosphate-buffered saline (PBS) until it had a final concentration of 200 mg/mL, followed by addition of the Ppy-PEI NC produced above to obtain a Ppy-PEI NC hydrogel with a final concentration of Ppy-PEI NC of 0.1-100 mg/mL.

### TEST EXAMPLE 1

### Morphology of Ppy-PEI NC Hydrogel

The Ppy-PEI NC hydrogel obtained in Preparation Example 1 was put in a 1.5 ml Eppendorf tube, which was then turned upside down and kept at room temperature (22-25° C.). Afterwards, the temperature was raised to 39-45° C. At room temperature, the Ppy-PEI NC hydrogel was in gel state and stayed above the inversed Eppendorf tube without the influence of gravity force. As the temperature was raised, the Ppy-PEI NC hydrogel was changed from gel state into solution state and flowed to the bottom of the Eppendorf tube. The state change of the Ppy-PEI NC hydrogel was reversible, and can change again from solution state to gel state as the temperature was dropped. Furthermore, gel-sol transition behavior of the Ppy-PEI NC hydrogel occurred around 35° C.

### TEST EXAMPLE 2

### Porous Structure of Ppy-PEI NC Hydrogel

FIG. 2 is a SEM diagram of the Ppy-PEI NC hydrogel obtained from the embodiment of the present disclosure. The Ppy-PEI NC hydrogel obtained from Preparation Example 1 was lyophilized to form a powder, whose structure was then observed by SEM. As shown in FIG. 2, compared with the pure gelatin hydrogel without Ppy-PEI NC, the Ppy-PEI NC hydrogel exhibited a significantly porous structure having a pore size around 0.1-0.2 mm, which was suitable for cellular growth. Therefore, the Ppy-PEI NC hydrogel of the present disclosure is suitable to serve as a biomimetic scaffold.

### TEST EXAMPLE 3

### Photothermal Properties of Ppy-PEI NC Hydrogel

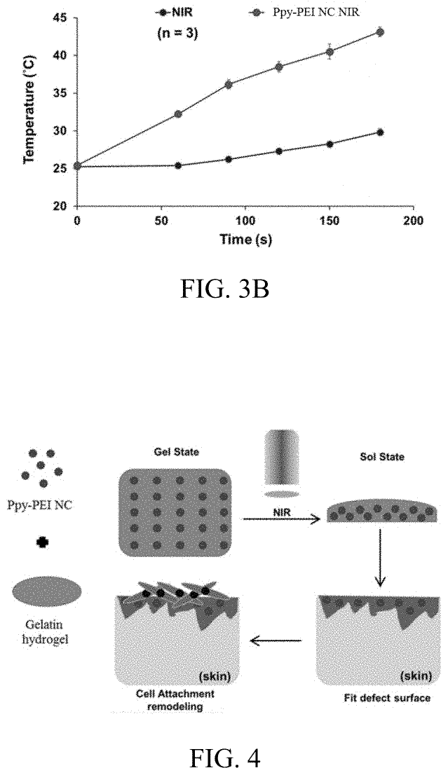

FIG. 3A shows thermal images of a near-infrared (NIR) irradiated Ppy-PEI NC hydrogel captured by a thermal camera, and FIG. 3B is a graph showing the quantitative temperature variation of FIG. 3A. Both the Ppy-PEI NC hydrogel obtained in Preparation Example 1 and the pure gelatin hydrogel without Ppy-PEI NC were exposed to remote NIR irradiation (808 nm) and detected by a thermal camera. As shown in FIG. 3B, the temperature of the hydrogel without the Ppy-PEI NC (NIR group) was slightly elevated from 25° C. to 29° C. in 180 sec. due to a lack of photothermal transduction efficiency. In contrast, after NIR treatment, the temperature of the Ppy-PEI NC hydrogel significantly increased from 25° C. to a hyperthermic temperature (43° C.), indicating that the Ppy-PEI NC indeed converts the absorbed near-infrared light into thermal energy.

FIG. 4 is a schematic flow diagram illustrating application of the Ppy-PEI NC hydrogel of the present disclosure onto a skin wound. The Ppy-PEI NC hydrogel was applied on the skin of a subject after receiving NIR irradiation. As mentioned above, the Ppy-PEI NC hydrogel was changed from gel state to solution state upon NIR irradiation, during which the absorbed NIR light was converted into thermal energy. The liquid Ppy-PEI NC hydrogel closely fitted the surface of damaged tissues, provided the cells with growth space, and thus facilitated wound healing.

### TEST EXAMPLE 4

### Cytotoxicity Assay of Ppy-PEI NC Hydrogel

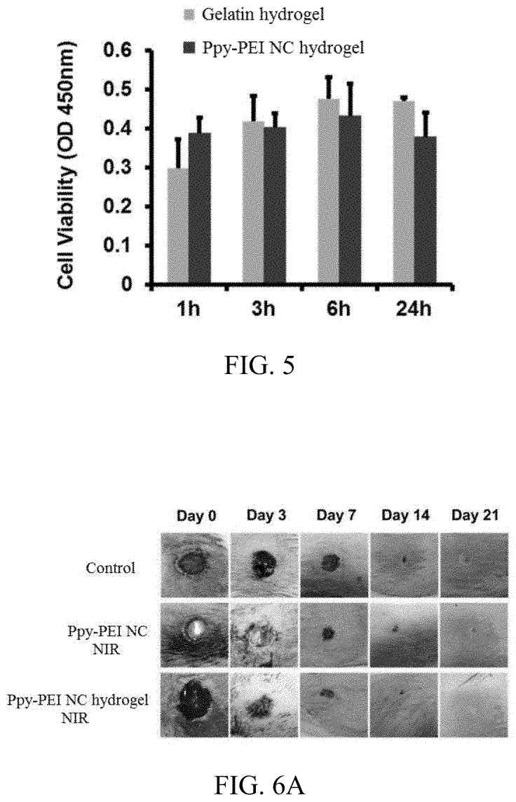

FIG. 5 shows the MTT assay results of the Ppy-PEI NC hydrogel according to an embodiment of the present disclosure. Equal volumes of the pure gelatin hydrogel and the Ppy-PEI NC hydrogel obtained from Preparation Example 1 were incubated in 5 mL high-glucose DMEM in 96-well plates and kept in an incubator at 37° C. and 5% CO₂ for 24 hr. Afterwards, 10 μL of the liquid extraction medium from each well was taken for indirect MTT cytotoxicity assay.

The L929 mouse fibroblast cells (5000-50000 cells/well, 0.1 mL) were seeded into a 96-well plate and then cultured for 24-48 h for appropriate attachment with growth. Then, extraction medium from both hydrogels (each 10 μL) was added into the 96-well plate and incubated for 24 hr. At the end, cellular viability was detected using an MTT kit and a microplate photometer (Multiscan FC, Mass., USA).

As shown in FIG. 5, no significant difference of cell viability was observed between the pure gelatin hydrogel (the control group) and the Ppy-PEI NC hydrogel of the present disclosure during 24 h of the assay, confirming that the Ppy-PEI NC hydrogel of the present disclosure has a similar biocompatibility with the gelatin hydrogel.

### TEST EXAMPLE 5

### In Vivo Study of Ppy-PEI NC Hydrogel

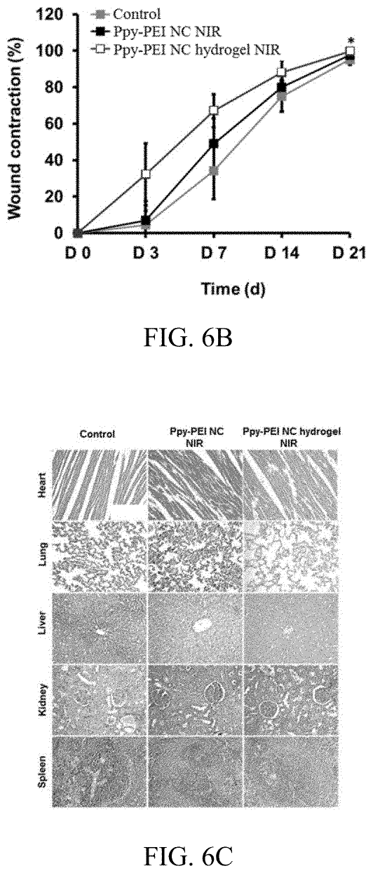

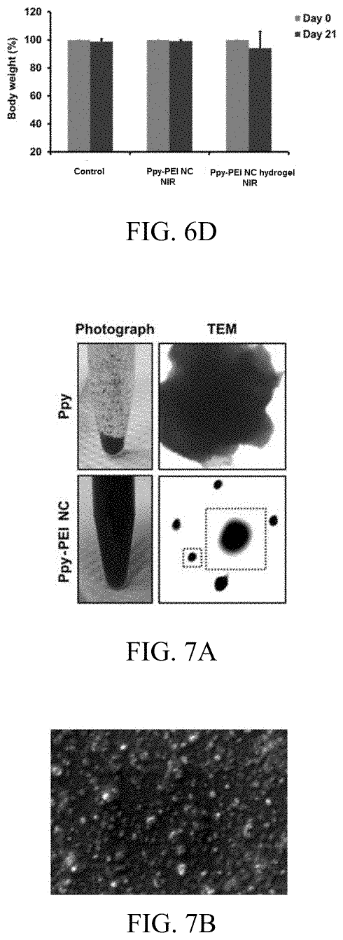

FIG. 6A shows the macroscopic images of rat skin wounds applied with the Ppy-PEI NC hydrogel of the present disclosure, and FIG. 6B shows the quantitative percentage of wound contraction (%).

During the course of the experiment, tested Wistar rats were randomly divided into three groups (n=3): a control group, a Ppy-PEI NC NIR group and a Ppy-PEI NC hydrogel NIR group. After each of the rats was anaesthetized and skin hairs on the back were removed, the skin was sterilized by using ethanol (70%), and then a 20-mm diameter circle was drawn. A circular cut was made around the drawn surface area of skin, and the skin was then carefully dissected to create a full thickness wound. The wound of the excision region was recorded instantly. After wound creation, the rats of the control group didn't receive any treatment; the rats of the Ppy-PEI NC NIR group were treated with Ppy-PEI NC with NIR irradiation at the wound sites; and the rats of the Ppy-PEI NC hydrogel NIR group were treated with Ppy-PEI NC hydrogel with NIR irradiation at the wound sites. The study was conducted for 21 days, and the parameters, i.e. wound size, wound area and percentage of wound contraction (%), assessed in each group (n=3) were recorded at different time points (day (d) 0, 3, 7, 14, and 21). The percentage of wound contraction (W %) was calculated according to the following formula:

W%=(W_{d0}−W_{dn})/W_{d0}×100

where W_{d0} means wound area at day 0, and W_{dn} means wound area at day n (n=0, 3, 7, 14, 21).

As shown in FIGS. 6A-6B, although at day 21, all groups of rats had shown almost complete wound closure, the Ppy-PEI NC hydrogel NIR group showed the best results at days 3, 7, and 14. Moreover, there was significant difference between the control group and the Ppy-PEI NC hydrogel NIR group at day 21 (p<0.05) in terms of wound contraction percentage, indicating that the Ppy-PEI NC hydrogel can effectively assist wound healing

After the observation and recording of the rats from three groups were completed at day 21, the rats were sacrificed under anesthesia, and the skin tissues, heart, lung, liver, kidney, and spleen at wound sites were collected, fixed in 2-50% buffered formalin, dehydrated using increasing concentrations of ethanol, and then embedded in paraffin. After sectioning, the samples were stained with hematoxylin and eosin stain (H&E Stain) for observation.

FIG. 6C shows images of histological sections of primary organs (heart, lung, liver, kidney, and spleen) of the rats from control and experimental groups. As shown in FIG. 6C, the histological analyses did not reveal any signs of inflammation and toxicity in control or experimental groups.

FIG. 6D is a graph showing percentage change in body weight (%). As shown in FIG. 6D, there was no significant body weight change observed among all the rats of the control and experimental groups between day 0 and day 21 of the experiment.

From FIGS. 6C-6D, it is evident that the Ppy-PEI NC hydrogel of the present disclosure shows biocompatibility in rats.

In this Test Example, the Ppy-PEI NC hydrogel NIR group showed the best wound contraction (%) at days 3, 7, and 14, indicating that the presence of gelatin hydrogel facilitates the growth of cellular tissue and wounding healing, which is contributed from the porous structure of the Ppy-PEI NC hydrogel that is suitable for cell growth and the energy conversion capability of Ppy-PEI NC to convert near-infrared light into heat and thus changing the Ppy-PEI NC hydrogel from gel state to solution state. In contrast, the Ppy-PEI NC without gelatin hydrogel remained in gel state after NIR irradiation, so it can't fit the uneven surface of wounds and can't provide a biomimetic scaffold to facilitate cell growth.

### PREPARATION EXAMPLE 2

### Preparation of Ppy-PEI NC

PEI (600 Da, 200 mg, purchased from Sigma-Aldrich) was dissolved in 20 mL of DI water to form a solution, into which a pyrrole monomer (12.5 μL, purchased from Sigma-Aldrich) was then added. The resulting solution was stirred for 0.2-3 h before addition of ferric chloride hexahydrate (12.5 mg/mL, 1 mL, purchased from Sigma-Aldrich). After 0.1-2 h of polymerization, the solution became black and then was removed free PEI and ferric ions, washed with DI water, and dried in an oven to obtain Ppy-PEI NC (20-1000 nm).

Different volumes of solvents can be added according to experimental needs to prepare various Ppy-PEI NC solutions of desired concentration.

### TEST EXAMPLE 6

### Dispersion of Ppy-PEI NC

FIG. 7A shows photographs and images of an aqueous Ppy-PEI NC solution and an aqueous Ppy solution captured by a general camera or transmission electron microscopy (TEM). The Ppy-PEI NC group was prepared by adding the Ppy-PEI NC (0.1-1 mL) obtained from Preparation Example 2 and water (0.1-1 mL) into an Eppendorf tube, followed by mixing with a shaker to form the aqueous Ppy-PEI NC solution. The Ppy group was the control group, which was prepared by adding polypyrrole (5-200 μL, purchased from Sigma-Aldrich) and water (0.01-0.5 mL) into an Eppendorf tube, followed by mixing with a shaker. As shown in the top row of photograph and image of FIG. 7A, Ppy lacks stability in the aqueous phase owing to its hydrophobicity, so that precipitation and aggregation behaviors occurred after the Ppy material was introduced into water. Once Ppy was stabilized with the polymeric PEI, Ppy-PEI NC dispersed homogeneously in water and formed a uniformly dark solution, as shown in the bottom row of photograph and image of FIG. 7A.

FIG. 7B shows an SEM image of the aqueous Ppy-PEI NC solution. As shown in FIG. 7B, the spherical shape of Ppy-PEI NC with a well-dispersed arrangement was possibly due to the smaller size together with stronger repulsive cationic electronic fields of each Ppy-PEI NC particle.

One of the main difficulties in generating dispersed nano-Ppy is the poor homogeneity of Ppy molecules in aqueous systems. Dispersion is worse for coating polymers without polar groups, as the polarity of the coating polymer has an impact on the dispersion of Ppy. In order to overcome this dispersion problem, the surface of Ppy is usually coated with a dispersion polymeric agent using different types of polymeric materials (e.g., polyethylenimine (PEI), heparin, fucoidan, hyaluronic acid, or glyco chitosan) previously exposed to polymerized pyrrole under mechanical stirring. The dispersion polymeric agent includes appropriately functionalized organic molecules which allow stabilization of Ppy polymeric molecules in aqueous solutions. In the present disclosure, the surface of Ppy is covered with PEI to form a Ppy-PEI nano-complex having a core-shell structure, thus enabling well dispersion of Ppy molecules in aqueous systems and expanding the applicability of Ppy.

### TEST EXAMPLE 7

### Surface Properties of Ppy-PEI NC

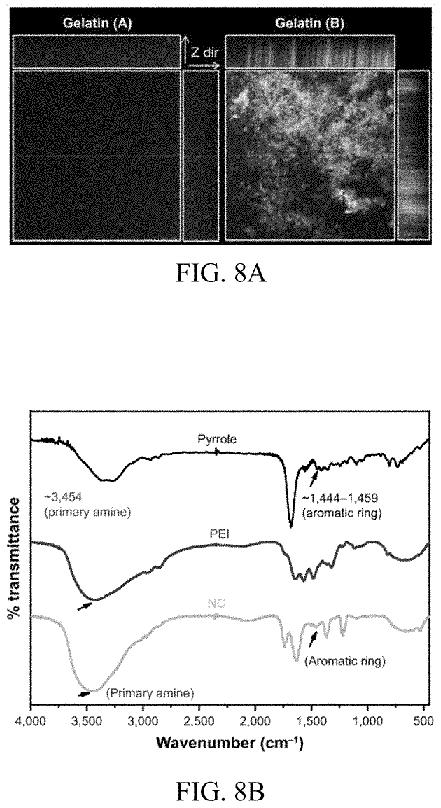

FIG. 8A shows images of Ppy-PEI NC incubated respectively with gelatin (A) and gelatin (B) hydrogels captured by confocal laser scanning microscopy (CLSM). Two equal amounts of the Ppy-PEI NC (0.002-200 mg/mL) prepared in Preparation Example 2 were added respectively into positively charged gelatin (A) hydrogel (10-1000 μL, Sigma-Aldrich) and negatively charged gelatin (B) hydrogel (10-1000 μL, Sigma-Aldrich), incubated for 0.1-2 hr, washed with PBS to remove Ppy-PEI NC unbound with the gelatin, and finally examined by CLSM. As shown in FIG. 8A, the cationic Ppy-PEI NC that accumulated on the negatively charged gelatin (B) hydrogel were greater than those on the positively charged gelatin (A) hydrogel, confirming that the Ppy-PEI NC of the present disclosure are positively charged and can be attracted by the negatively charged gelatin (B) hydrogel.

In solid cancerous biology, neutrophils act as a dominant cell species in the tumorous tissue region surrounding infiltration. The neutrophils with tumor surrounding tissue are the cells with cationic charged peptides/substances covering the surface. Besides, the anionic charges were found to be generated from the huge amount of lactate secretions, a recognized feature of an entirely metabolically active cancerous cell line. Thus, this different surface charged feature indicated that targeting negative surface charges of cancer cells by cationic PEI coated Ppy-PEI NC particles can provide efficient targeting treatment toward cancer cells with huge amount of anionic charges.

FIG. 8B is a graph showing the analyzed result of the Ppy-PEI NC by Fourier transform infrared (FTIR) spectroscopy. To further demonstrate the core-shell structure formed by the covalent bonding between Ppy and PEI, FTIR was used to study the chemical structure of the Ppy-PEI NC obtained from Preparation Example 2. As shown in FIG. 8B, Ppy-PEI NC exhibited characteristic peaks around 1444˜1459 cm⁻¹ originated from stretching vibrations of aromatic rings of Ppy as well as characteristic peaks around 3,454 cm⁻¹ originated from the primary amine of PEI.

### TEST EXAMPLE 8

### Cellular Uptake of Ppy-PEI NC and ROS/H₂O₂ Detection of NIR Irradiated Ppy-PEI NC

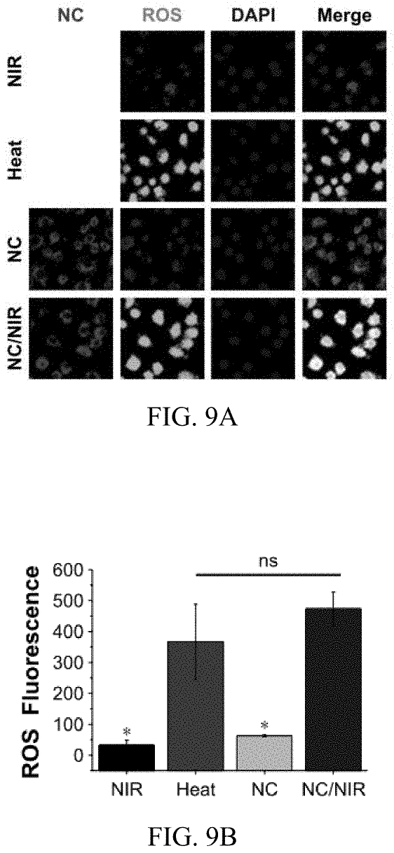

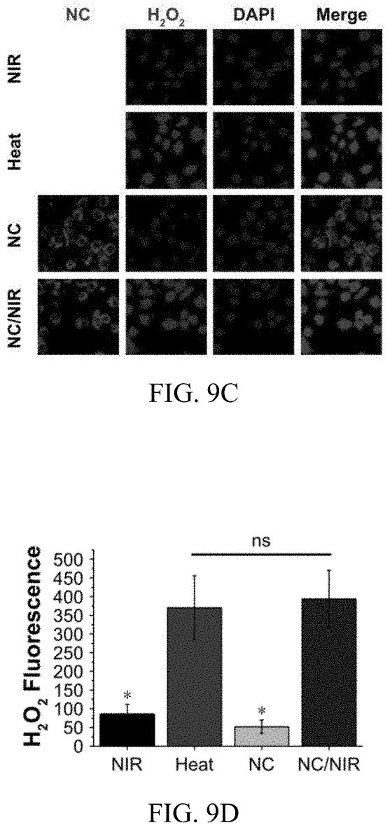

FIG. 9A shows fluorescent confocal laser scanning microscopy images illustrating cell endocytosis and detected ROS; FIG. 9B is a graph showing statistical analysis results of ROS fluorescence intensity in FIG. 9A; FIG. 9C shows fluorescent confocal laser scanning microscopy images illustrating cell endocytosis and detected H₂O₂; and FIG. 9D is a graph showing statistical analysis results of H₂O₂ fluorescence intensity in FIG. 9C; wherein the NIR group had been irradiated by NIR; the Heat group had been heated; the NC group had been added with Ppy-PEI NC but not irradiated by NIR; and the NC/NIR group had been added with Ppy-PEI NC and irradiated by NIR.

In this Test Example, lung cancer cells H460 (from ATCC® HTB-177™; American Type Culture Collection (ATCC), Manassas, Va., USA) were seeded into the confocal dishes, and then these dishes were kept in a cell incubator at 37° C. and 5% CO₂ overnight. Afterwards, the cells in the dish were kept in Hank's balanced salt solution (HBSS) for 1 hr., and then incubated with or without Cy5 labeled Ppy-PEI NC (0.002-200 mg/mL) for 1 hr. To create a hyperthermia environment, the dishes was placed in a water bath incubator as an additional heat source for 0.1-4 hr. The cells in the dishes were then flushed 3 times with PBS and stained by 4′,6-diamidino-2-phenylindole (DAPI), dichlorofluorescin diacetate (DCFDA, ROS dye), and Amplex Red (hydrogen peroxide dye) to elucidate biocellular interactions. Fluorescent results were visualized through CLSM. The fluorescence signal intensity was quantitatively measured by ImageJ software.

As shown in FIGS. 9A to 9D, the group treated with NIR alone and the group received with only Ppy-PEI NC generated few ROS but hydrogen peroxide was clearly observed. However, the group treated with an additional heat source or the group of Ppy-PEI NC with NIR treatment generated significant ROS and hydrogen peroxide, as clearly analyzed by CLSM and quantitatively measured by ImageJ software.

In this Test Example, it has been demonstrated that the cellular uptake of Ppy-PEI NC into the cancer cells is through clathrin-dependent pathways. The dimension-dependent uptake of various biomaterials in diverse cellular lines has been studied with maximum cell internalization at a nano-material core dimension in a range of around 60-400 nm, which indicates that the Ppy-PEI NC particles having a size ranging from 10-1000 nm disclosed herein can be easily uptaken by the cancer cells. Combined with the characteristic of positively charged surface, which promotes the attachment of Ppy-PEI NC onto the cancerous cells, the Ppy-PEI NC of the present disclosure are useful in cancer treatment.

### TEST EXAMPLE 9

### MTT Assay of Ppy-PEI NC

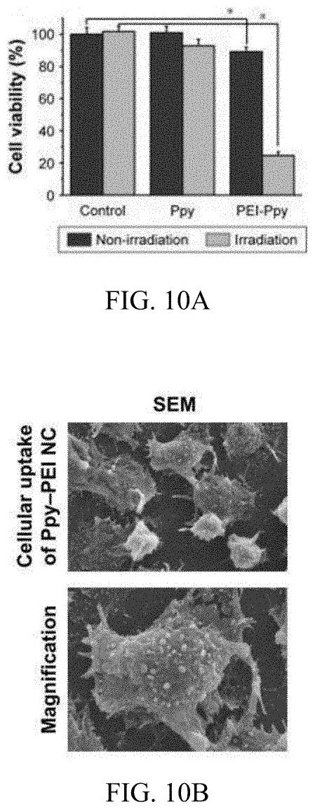

FIG. 10A is a graph showing statistical analysis results of MTT assays; and FIG. 10B shows SEM images of cancer cells. In this test example, the NCI-H460 cells were cultured in 96-well plates with cell growth medium at 0.1-10×10⁴ cells/well and 37° C. with 5% CO₂ overnight. The tested cancer cells were flushed twice with HBSS and then supplemented with 200 μL of Ppy or Ppy-PEI NC dissolved in HBSS (0.15-150 mg/mL). After 1 hr, the 96-well plates were irradiated with or without NIR light (0-60 min under 2 W/cm²), followed by flushing twice with PBS. Next, 20 μL of MTT solution (5 mg/mL dissolved in PBS, Sigma-Aldrich) was added into each well, followed by subsequent cell culture for another 1-4 h at 37° C. in an incubator with 5% CO₂. Subsequently, the medium was withdrawn, and dimethyl sulfoxide (DMSO) was added to incubate for 10-30 min The absorbance at 490-570 nm was measured by an ELISA reader.

As shown in FIG. 10A, the control group was not added with Ppy or Ppy-PEI NC. After NIR irradiation, the cell viability of the control group was not dropped, indicating that the near-infrared light is not cytotoxic to cells. As to the Ppy-PEI NC group, the cell viability is over 80% without NIR irradiation, indicating that Ppy-PEI NC has relatively low cytotoxicity. Cells may show a poor adhesion onto hydrophobic and aggregated materials (Ppy), resulting that the low cytotoxic effect after washing and NIR treatment. However, the group that received Ppy-PEI NC with NIR treatment displayed a cytotoxic effect compared to the control group.

FIG. 10B shows SEM images of Ppy-PEI NC attached onto cancer cells. As shown in FIG. 10B, Ppy-PEI NC particles are observed on the surfaces of cells due to cellular uptake of Ppy-PEI NC.

### TEST EXAMPLE 10

### Apoptosis Induced by Ppy-PEI NC

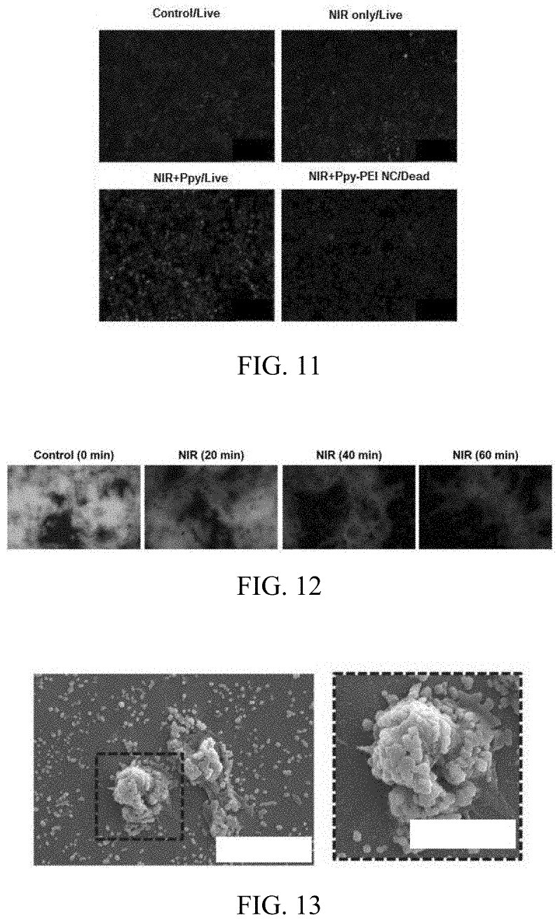

FIG. 11 shows images of cells by fluorescence microscopy. In this test example, the NCI-H460 cells were cultured in dishes kept in an incubator at 37° C. with 5% CO₂ overnight. Afterwards, the cells were flushed twice with HBSS and then supplemented with 0-200 μL of Ppy or Ppy-PEI NC dissolved in HBSS (0.15-150 mg/mL). After 1 hr, the cells were irradiated with NIR light (1-100 min under 2 W/cm²) except the control group, followed by flushing twice with PBS. The cells were then stained by live/dead viability/cytotoxicity assay kit (Molecular Probes, Eugene, Oreg., USA) for 30 min , and detected by fluorescence microscopy.

As shown in FIG. 11, a high viability was detected within similar cellular morphologies observed in the untreated (control), NIR only, and Ppy with NIR groups. In contrast, the group that received Ppy-PEI NC and then non-invasive NIR treatment exhibited significant cytotoxicity, indicating that the NIR-treated Ppy-PEI NC provided by the present disclosure induces cancer cell apoptosis, thus facilitating cancer therapy.

### PREPARATION EXAMPLE 3

### Preparation of Cy5-Ppy-PEI NC

PEI (600 Da, 200 mg, purchased from Sigma-Aldrich) was dissolved in 20 mL of DI water to form a solution, into which a pyrrole monomer (12.5 μL, purchased from Sigma-Aldrich) was then added. The resulting solution was stirred for 0.2-3 h before addition of ferric chloride hexahydrate (12.5 mg/mL, 1 mL, purchased from Sigma-Aldrich). After 0.2-3 h of polymerization, free PEI and ferric ions were removed, and then DI water washing and oven drying were performed to obtain Ppy-PEI NC (20-1000 nm). To facilitate observation, a Cy5-NHS (Cy5-N-hydroxysuccinimide) fluorescent dye was mixed with the Ppy-PEI NC (0.1-200 mg/mL) obtained above under a pH value of 7.4 at a temperature of 4-37° C. for 4-24 hr, followed by dialysis in DI water for 2-7 days to remove unlabeled derivatives, resulting in labeled Cy5-Ppy-PEI NC (0.1-200 mg/mL).

### TEST EXAMPLE 11

### In Vitro Anti-Clot Effect of Ppy-PEI NC

FIG. 12 shows CLSM images of colt morphology, demonstrating the in vitro anti-clot effect of Ppy-PEI NC with NIR irradiation.

To test photo-thermal effect on in vitro anti-clot, the Alexa Fluor 647-conjugated fibrinogen (purchased from Sigma-Aldrich) was dissolved in a Tris-HCl (5-5000 mM)-NaCl (0.14-100 mM) buffer at pH 7.4 to form a fibrinogen solution (0.01-1000 mg/mL). Clot formation (polymerized fibrin) is initiated by adding thrombin (0.1-5 U/mL) and CaCl₂ (0.25-100 mM) to the fibrinogen solution, followed by incubation at 37° C. for 1 h. To investigate photo-thermal ablation against fluorescent clots, the Ppy-PEI NC (0.5-100 mg/mL) was added into the fibrinogen solution (0.9-100 μL) with thrombin before adding CaCl₂ except the control group. To simulate the physiological environment, the fluorescent clots were put on parallel slides and exposed to additional shear forces from a PBS flow. The tested samples were then exposed under NIR irradiation (2.0 W/cm²) for 0.1-3 h and examined under a confocal microscope to observe the change in density of fibrin.

As shown in FIG. 12, shear-induced depletion of fibrin from the thrombus matrix was observed in confocal images after NIR irradiation. The untreated control evidenced high-density colt morphology even under an additional shear force. In contrast, once the NIR irradiation (20, 40 or 60 min) and shear forces were applied, the morphology changed from an intact to a loosen type.

### TEST EXAMPLE 12

### In Vivo Biodistribution and Histological Test Showing Accumulation of Ppy-PEI NC at the Thrombus Sites Via Macrophages in Live Animals

FIG. 13 shows SEM images of macrophages co-localized with the positively charged Cy5-Ppy-PEI NC. Macrophages, RAW 264.7 (ATCC® TIB-71™), are kept in DMEM supplemented with 10% FBS). Test RAW 264.7 seeded into confocal dish (5000 cell/dish) were washed by Hank's Balanced Salt Solution (HBSS) and the test Ppy-PEI NC in HBSS added for 2 h. Afterwards, the cells were stained using macrophage antibody (FITC-F4/80 PE), DAPI and a ROS indicator, DCFH-DA. As shown in FIG. 13, the positively-charged Cy5-Ppy-PEI NC were found to interact with negatively charged macrophages' cell membrane and underwent extensive phagocytosis. After cell internalization, most Cy5-Ppy-PEI NC were observed to concentrate into the lysosomal compartments nearby the cell nuclei.

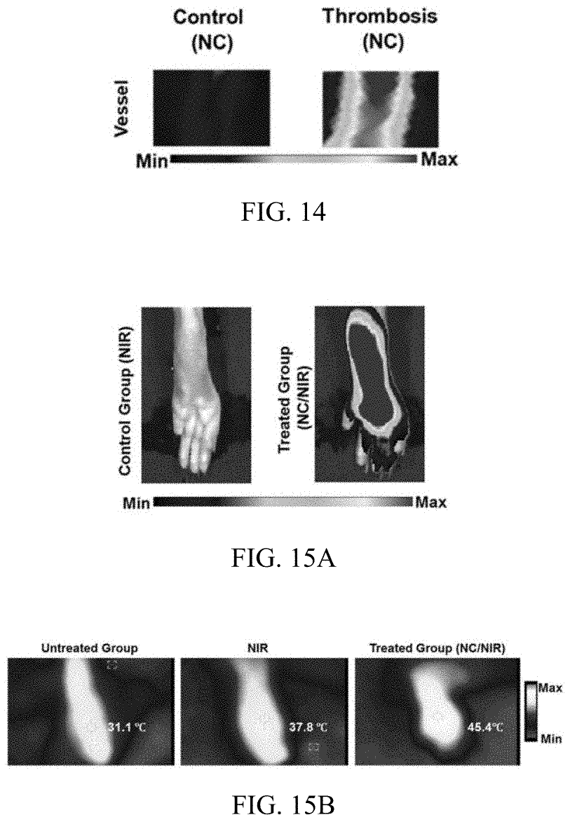

FIG. 14 shows IVIS images of the thrombus sites at femur veins of Wistar rats. Wistar rats (250-350 g, BioLASCO) were divided into a control group and a Ppy-PEI NC group. The rats are anesthetized by using 2-4% isoflurane and their femur veins are surgically exposed. Next, the thrombus of femur veins is generated by covering with a filter paper containing 0-50% ferric chloride for 0-30 min. In the biodistribution study, the Cy5-Ppy-PEI NC (0.1-200 mg/mL) obtained from Preparation Example 3 were systemically administrated by cardiac injection. After administration for 0-60 min, the rats were sacrificed and the thrombus sites of femur veins were fluorescently observed through in vivo imaging system (IVIS).

As shown in FIG. 14, the IVIS images clearly revealed that the Cy5 signals from systemically administered Cy5-Ppy-PEI NC specifically accumulated at the thrombus sites of femur veins, compared to the control group without Cy5-Ppy-PEI NC treatment. It was expected that the Cy5-Ppy-PEI NC would be detected and then uptaken by macrophages in blood due to cellular internalization after systemic administration through cardiac injection. Macrophages, accumulating around the thrombus sites due to immune response, can therefore serve as a thrombus targeting carrier system for photo-thermal anti-clot treatment.

### TEST EXAMPLE 13

### In Vivo Photothermal Properties of Ppy-PEI NC

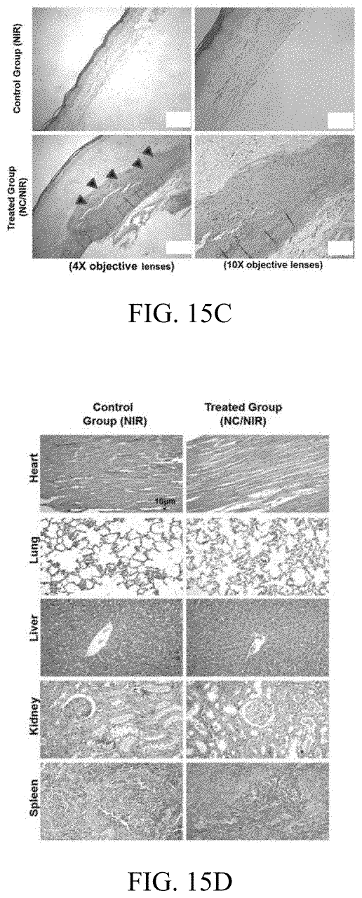

FIG. 15A shows IVIS images of rats' feet; FIG. 15B shows images of rats' feet by a thermal camera; FIG. 15C shows images of tissue sections by optical microscopy; and FIG. 15D shows images of organ sections by optical microscopy.

In the present Test Example, Wistar rats (250-350 g, BioLASCO) were divided into a control (NIR) group and a Ppy-PEI NC group. For the Ppy-PEI NC group, the Cy5-Ppy-PEI NC (0.1-200 mg/mL, 0.01-1 mL) prepared from Preparation Example 3 was subcutaneously injected to rats' feet, followed by NIR irradiation (2 W/cm²) for 0-60 min. As to the control (NIR) group, the rats were subject to NIR irradiation only without Cy5-Ppy-PEI NC administration. Afterwards, the injection sites of the rats were observed through in vivo imaging system (IVIS) and the thermal camera. After 2-7 days, the test rats were sacrificed, and their skin tissues at the injection sites as well as heart, lung, liver, kidney, and spleen were gathered, fixed in 2-50% buffered formalin, dehydrated using increasing concentrations of ethanol, and then embedded in paraffin. After sectioning, the samples were stained with hematoxylin and eosin stain (H&E Stain) for observation by optical microscopy.

As shown in FIG. 15A, the Cy5 labeled Ppy-PEI NC was clearly detected by IVIS compared with control (NIR). As shown in FIG. 15B, after NIR irradiation, the local temperature at the injection sites of the Ppy-PEI NC group was increased to the hyperthermia range (42-46° C.). However, the local temperature of the other group (untreated and NIR only) was below 38° C. without significant temperature rise. As shown in FIG. 15C, histological examination was also performed to investigate the ability of in vivo macrophage recurrence by the Ppy-PEI NC. Compared to the NIR control, the rats which received the Ppy-PEI NC with NIR proved photo-thermal treatment showed that structures with a macrophage-like morphology interacted with the implanted Ppy-PEI NC 3 days after implantation visualized under a light microscopy. As shown in

FIG. 15D, no adverse effect such as inflammation was observed in the tested organs (soft tissue including heart, lung, liver, kidney and spleen) of the control and Ppy-PEI NC groups.

The in vivo histological outcomes suggested that all the tested organs in the tested rats taking the Ppy-PEI NC of the present disclosure showed no abnormalities compared with NIR control group. In addition, such local photothermal treatment should not cause damage to the vascular endothelium and vascular wall, because the blood flow near the thrombus may weaken the locally formed thermal response and prevent it from spreading to the vascular wall. Therefore, the Ppy-PEI NC of the present disclosure can be applied to the treatment of thrombus without causing damage to the vascular endothelium and vessel wall at the patient's thrombus.

### PREPARATION EXAMPLE 4

### Preparation of Ppy-PEI NC-PE Fiber Constructed Membrane (Ppy-PEI NC-PEFM)

Using the dip-coating method, a PE fiber constructed membrane (PEFM) was soaked in the aqueous Ppy-PEI NC solution (0.2-100 mg/mL) prepared from Preparation Example 2 for one day and washed three times using DI water to obtain a Ppy-PEI NC-PE fiber constructed membrane (Ppy-PEI NC-PEFM). Similarly, another PE-fiber-constructed membrane was soaked in an aqueous Cy5-Ppy-PEI NC solution (0.2-100 mg/mL) prepared using Cy5-Ppy-PEI NC of Preparation Example 3 for one day and washed three times using DI water to obtain a Cy5-Ppy-PEI NC-PE fiber constructed membrane (Cy5-Ppy-PEI NC-PEFM).

### TEST EXAMPLE 14

### Photothermal Property and Washability of PEFM and Ppy-PEI NC-PEFM

The PEFM and Ppy-PEI NC-PEFM were washed with DI water, and then irradiated under NIR lamp (0.1-100 min) The changes in temperature were measured using a thermal camera (HuaZhi Electronic Technology Co., Ltd., Zhengzhou, China) or a thermocouple (Lutron TM-925, USA). The spectroscope was obtained with a fiberoptic spectrometer (Ocean Optics HR 2000+) to check the wavelength of light irradiated from the NIR lamp. To microscopically visualize the distribution of the Ppy-PEI NC onto the PEFM, cy5-NHS-ester was made to covalently conjugate on the Ppy-PEI NC.

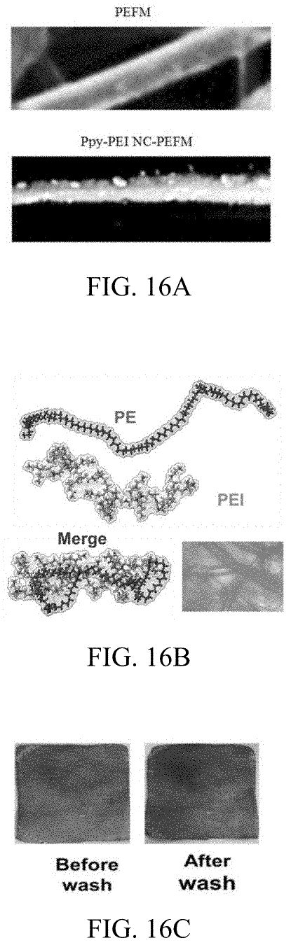

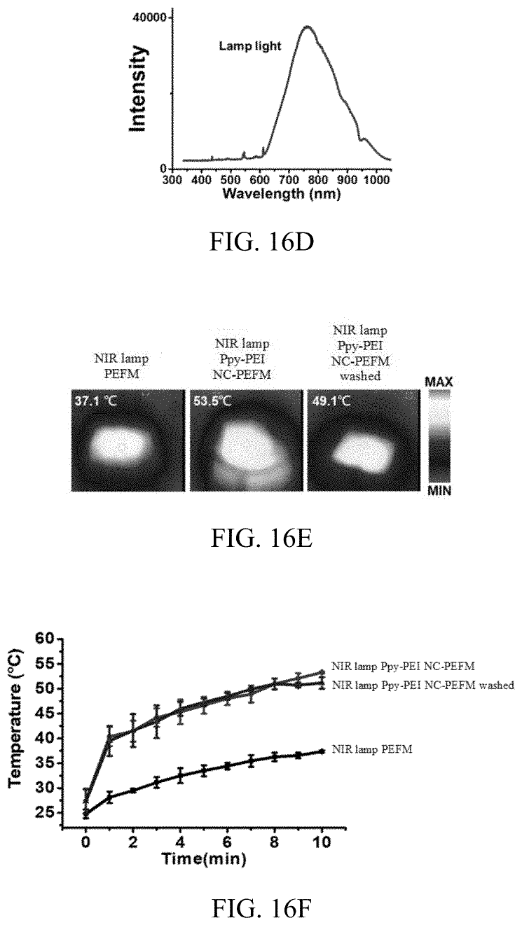

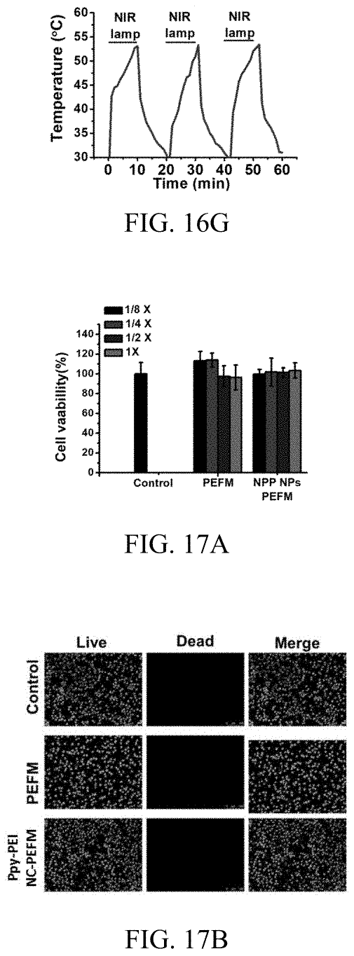

At high magnified field, the SEM data showed a number of Ppy-PEI NC particles attached to the PE fibers of the Ppy-PEI NC-PEFM (FIG. 16A), compared to the smooth surface of PEFM. The possible mechanism is that the PEI of shelled Ppy-PEI NC can be covalently grafted onto the surface of the PE fibers. The optical microscopic data showed that a dark color distribution on the fibers of Ppy-PEI NC-PEFM was observed, implying that the Ppy-PEI NC were attached onto the fiber surface (FIG. 16B). To understand this mechanism, we performed molecular dynamics (MD) simulations to study the PEI-mediated interaction with PE fibers of PEFM. The MD-simulation data showed that the binding affinity of PEI with PE was negative value of Kcal/mol, suggesting that the PEI of Ppy-PEI NC can bind onto the PE fibers of PEFM. No color change was observed in photographic data of Ppy-PEI NC-PEFM before and after washing (FIG. 16C), suggesting that the anti-wash ability of Ppy-PEI NC-PEFM was achievable.

Approximately 50% of the sunlight incident on the surface of the earth is in the NIR-wavelength range, i.e. wavelength greater than 650 nm. The spectroscope was used for checking the NIR lamp light, which mimic sunlight. The spectroscopic data showed that wavelength of NIR lamp light was around 600 to 900 nm (FIG. 16D). As shown in FIG. 16E and FIG. 16F, the photothermal effects of washable Ppy-PEI NC-PEFM exhibited higher NIR absorbance than that of PEFM, as well as more efficient photothermal conversion under the NIR lamp radiation. Also, Ppy-PEI NC-PEFM can tolerate thorough washing processes and still offer significant photo-stability, even upon repeated NIR irradiating instances (FIG. 16G). Therefore, the Ppy-PEI NC-PEFM of the present disclosure is a photothermal conversion textile that can selectively absorb and convert the NIR-containing solar light into thermal energy, thereby effectively enhancing the thermal-generation properties of the textile.

### TEST EXAMPLE 15

### In-Vitro Biocompatibility and Antibacterial Mechanism

Mouse L929 cells were grown in 5-20%-FBS DMEM with 0.1-10% penicillin/streptomycin. The cells were maintained at 1-20% CO₂ and at 37° C., under aseptic conditions until the L929 cells reached confluence. To evaluate the biocompatibility of both PEFM and Ppy-PEI NC-PEFM, a conventional extracting method was adopted. In brief, the tested membranes were sterilized for 0-36 h in 50-90% ethanol and irradiated overnight under UV light. Subsequently, the tested membranes (6.5 cm×6.5 cm) were immersed into a cell-growth medium for 0-36 h at 37° C. for attaining extraction. The suspensions of the L929 cells were seeded for one day onto a 96-well plate for culturing.

Thereafter, 0.001-1 mL of the extracted medium was supplemented into all the 96 wells (cells) and cultured for one day. It was examined using the MTT method. Subsequently, the optical density of each of the 96 wells was recorded at 570 nm using a microplate reader (Molecular Devices, USA). The assay data for different experimental groups were measured and compared using statistical analysis. The viability of the L929 cells was also assessed using the ethidium homodimer-1 (EthD-1, for staining dead cells) and calcein AM (for staining green living cells) dyes, followed by detection using a fluorescent microscope.

Escherichia coli (E. coil) bacteria were obtained and maintained in a bacteria incubator. To observe the attachment of the bacteria on the surface of the tested membranes, the bacterial suspensions were blended at different formulations (PEFM and Ppy-PEI NC-PEFM), washed with PBS and then stained using Hoechst for performing the fluorescent microscopic assay. To further estimate the photothermal-bactericidal activity, the tested membranes absorbing bacteria (without Hoechst staining), as mentioned previously, upon NIR lamp treatment (0.1-360 min) were determined.

As depicted in FIG. 17A and FIG. 17B, the MTT and live/dead data indicated that the cytotoxicity effect of either PEFM or Ppy-PEI NC-PEFM was negligible, suggesting that Ppy-PEI NC-PEFM is non-toxic and biocompatible as a multi-functional textile.

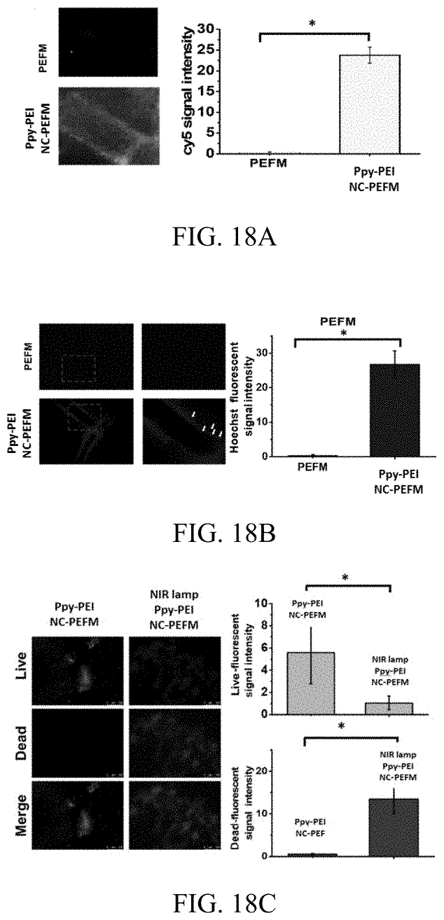

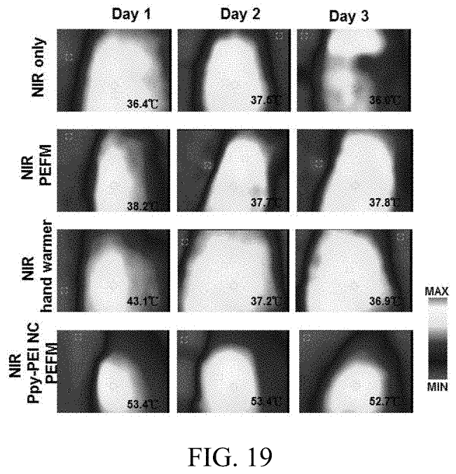

PEI has been examined for its strong binding with bacteria via electrostatic bio-interactions. The cy5 fluorescent signal indicated Cy5-Ppy-PEI NC distribution on the PEFM (Cy5-Ppy-PEI NC-PEFM), compared with the PEFM without Cy5-Ppy-PEI NC (FIG. 18A). The PEFM or Ppy-PEI NC-PEFM was tested for effectively capturing gram-negative E. coli bacteria. As depicted in FIG. 18B, the fluorescence microscopic data indicated that after 0.1-360 h of incubation on the surface of PEFM, the amount of viable E. coil cells was negligible. In contrast, the presence of PEI amino groups in Ppy-PEI NC-PEFM exhibited a cationic property and therefore the surface of Ppy-PEI NC-PEFM could capture strong negatively charged bacteria through the mechanism of electrostatic interaction, as depicted in FIG. 18B.

After 0-360 min of NIR lamp irradiation on the Ppy-PEI NC-PEFM incubated with bacteria, the fluorescence microscopic result showed that the retained bacteria could be significantly eradicated. Therefore, it was confirmed that Ppy-PEI NC-PEFM possesses the ability of photothermal ablation of microorganisms on the textile under sunlight (FIG. 18C).

### TEST EXAMPLE 16

### In-Vivo Photothermal and Toxicity Study

To facilitate the observation of in-vivo photothermal effect of the tested materials, PEFMs or Ppy-PEI NC-PEFMs were individually placed onto the backs of anesthetized rats, followed by irradiation under a NIR lamp. The thermal change on the back tissues in vivo were measured after 5 min NIR lamp irradiation at days 0, 1, and 2. The temperature distribution of the treated animals (NIR lamp irradiation alone, NIR lamp irradiation plus PEFM, NIR lamp irradiation plus Ppy-PEI NC-PEFM, or NIR lamp irradiation plus commercial hand warming [Kobayashi Pharmaceutical Co., Ltd., Japan]) were obtained using a thermal camera (A-BF RX-300). To further understand the vasodilation caused by the photothermal effect, the Ppy-PEI NC-PEFMs were placed on rabbit ears and, subsequently, irradiated under the NIR lamp. The images of rabbit's ear were obtained by a camera. At day 2 after treatment, the skin (treated region and surrounding skin), heart, liver, lung, spleen, and kidney of all the tested groups were harvested via scarification of the animals in order to perform histopathological analysis, including hematoxylin and eosin staining.

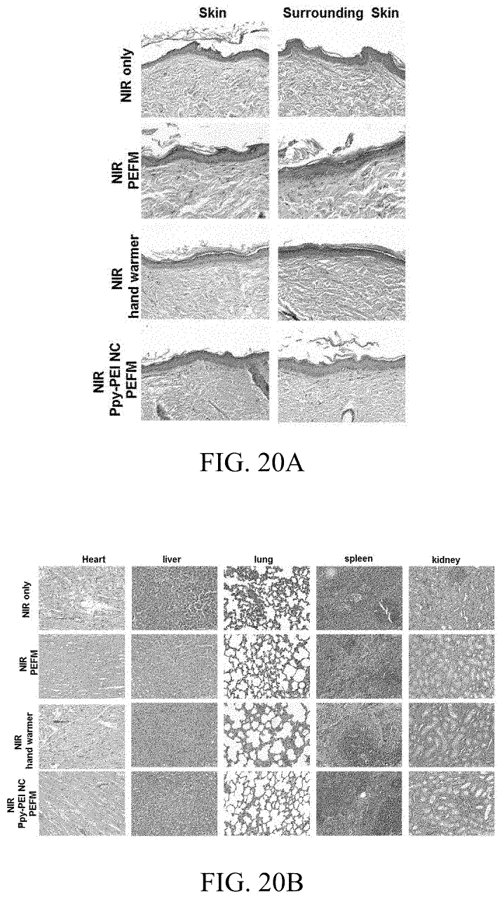

As shown in FIG. 19, the group of NIR lamp irradiation plus Ppy-PEI NC-PEFM demonstrated higher temperature increase compared with other groups, logically suggesting the use as a photothermal cloth at high altitudes or latitudes (below −0° C. with strong solar irradiation). Owing to the photothermal effect of the Ppy-PEI NC-PEFM group treated with NIR lamp irradiation, the generated hyperthermia produced an improved blood flow, leading to increased oxygenation, perfusion, and vasodilation, all of which were observed via the rabbit-ear study. Furthermore, the detailed histology study of the tested rats showed no tested group had any harmful in vivo toxicity (see FIG. 20A and FIG. 20B). However, in some cases, frostbite occurred upon exposure to freezing temperatures, damaging the tissues or skin. It has been evidenced that Ppy-PEI NC-PEFM showed better photostability and higher photothermal conversion efficiency under NIR lamp irradiation than those shown by the other groups. Furthermore, the in-vitro and in-vivo findings suggested the biocompatibility, antibacterial property, and photothermal effect of Ppy-PEI NC-PEFM after its long-term monitoring as fabric applications.

The conductive polymer material of the present disclosure can also be used as a technology platform in the field of thermotherapy-related physical therapy. For example, the photothermal patch combined with a medical infrared light source can be used to stimulate the acupuncture points and produce a therapeutic effect like the traditional Chinese medicine cupping, acupuncture, flying needle treatment, etc. Because the conductive polymer is cheap, biodegradable, and can perform stable photothermal effects after repeated irradiations, it can be more economical and environmentally friendly to replace the existing equipment and provide improved physical therapy effect.

Although the present invention has been explained in relation to its preferred embodiment, it is to be understood that many other possible modifications and variations can be made without departing from the spirit and scope of the invention as hereinafter claimed.

What is claimed is:

1. A complex having a core-shell structure, comprising:
a core; and
a shell layer covering a surface of the core;
wherein the core is made of polypyrrole.

2. The complex of claim 1, wherein the shell layer is made of a material selected from athe group consisting of polyethylenimine (PEI), heparin, fucoidan, hyaluronic acid, glyco chitosan, and a combination thereof.

3. The complex of claim 1, wherein the shell layer is made of polyethylenimine.

4. The complex of claim 1, wherein the complex has a size ranging from 10 nm to 1500 nm.

5. The complex of claim 1, wherein a weight ratio of the shell layer to the core ranges from 1500:500 to 100:4.

6. A method for treating thrombosis, comprising: administrating to a subject in need thereof an effective amount of a complex having a core-shell structure, wherein the complex comprises:
a core; and
a shell layer covering a surface of the core;
wherein the core is made of polypyrrole.

7. The method of claim 6, wherein the shell layer is made of a material selected from the group consisting of polyethylenimine (PEI), heparin, fucoidan, hyaluronic acid, glyco chitosan, and a combination thereof.

8. The method of claim 6, wherein the shell layer is made of polyethylenimine.

9. The method of claim 6, wherein the complex has a size ranging from 10 nm to 1500 nm.

10. A method for treating cancer, comprising: administrating to a subject in need thereof an effective amount of a complex having a core-shell structure, wherein the complex comprises:
a core; and
a shell layer covering a surface of the core;
wherein the core is made of polypyrrole.

11. The method of claim 10, wherein the shell layer is made of a material selected from the group consisting of polyethylenimine (PEI), heparin, fucoidan, hyaluronic acid, glyco chitosan, and a combination thereof.

12. The method of claim 10, wherein the shell layer is made of polyethylenimine.

13. The method of claim 10, wherein the complex has a size ranging from 10 nm to 1500 nm.

14. The method of claim 10, wherein the cancer is lung cancer.

15. A composition, comprising:
a complex having a core-shell structure, comprising:
a core made of polypyrrole; and
a shell layer covering a surface of the core; and
a polymer.

16. The composition of claim 15, wherein the shell layer is made of a material selected from the group consisting of polyethylenimine (PEI), heparin, fucoidan, hyaluronic acid, glyco chitosan, and a combination thereof.

17. The composition of claim 15, wherein the shell layer is made of polyethylenimine.

18. The composition of claim 15, wherein the complex has a size ranging from 10 nm to 1500 nm.

19. The composition of claim 15, wherein the polymer is a thermally sensitive hydrogel.

20. The composition of claim 15, wherein the polymer is a binder.

21. A textile, comprising:
a fiber; and
a complex having a core-shell structure and attached to the fiber, comprising:
a core made of polypyrrole; and
a shell layer covering a surface of the core.

22. The textile of claim 21, wherein the fiber is a polyethylene (PE) fiber.

23. The textile of claim 21, wherein the shell layer is made of a material selected from the group consisting of polyethylenimine (PEI), heparin, fucoidan, hyaluronic acid, glyco chitosan, and a combination thereof.

24. The textile of claim 21, wherein the shell layer is made of polyethylenimine.

25. The textile of claim 21, wherein the complex has a size ranging from 10 nm to 1500 nm.

26. The textile of claim 21, wherein a weight ratio of the core to the shell layer ranges from 1500:500 to 100:4.
